(19) United States
(12) Patent Application Publication
(10) Pub. No.: US 2005/0048585 A1
(43) Pub. Date: Mar. 3, 2005

(54) Methods and compositions for modulating NF-AT transcription factor

(75) Inventors: Suhaila White, San Diego, CA (US); Sumit Chanda, La Jolla, CA (US); Jeremy S. Caldwell, Cardiff, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, a Delaware Limited Liability Company, Hamilton (BM)

(21) Appl. No.: 10/732,894

(22) Filed: Dec. 10, 2003

Related U.S. Application Data
(60) Provisional application No. 60/433,389, filed on Dec. 13, 2002.

(51) Int. Cl. G01N 33/53; G01N 33/567

(52) U.S. Cl. 435/7.2

(57) ABSTRACT

This invention provides novel NF-AT-modulatory polypeptides. The invention also provides methods for screening modulators of NF-AT. The methods comprise first screening test agents for modulators of an NF-AT-modulatory polypeptide and then further screening the identified modulating agents for modulators of NF-AT. The invention further provides methods and pharmaceutical compositions for modulating NF-AT bioactivities in a cell and for treating diseases and conditions mediated by abnormal NF-AT activities.

### CROSS-REFERENCE TO RELATED APPLICATIONS

This application claims the benefit of priority to U.S. Provisional Patent Application Ser. No. 60/433,389 (filed Dec. 13, 2002), the disclosure of which is incorporated herein by reference in its entirety and for all purposes.

### FIELD OF THE INVENTION

The present invention generally relates to methods for identifying modulators of NF-AT transcription factors and therapeutic applications of such modulators. More particularly, the invention pertains to novel NF-AT modulators that regulate bioactivities and cellular level of NF-AT, and to methods of using such modulators to modulate transcription-regulating activities or cellular level of NF-AT in a subject.

### BACKGROUND OF THE INVENTION

Nuclear Factor of Activated T cells (NF-AT) is a family of at least four related transcription factors (NF-AT1, NF-AT2, NF-AT3, and NF-AT4) which plays a key role in regulating lymphokine gene expression (see e.g., Serfling et al., Biochim Biophys Acta 1498: 1-18, 2000; and Lopez-Rodriguez et al., Cold Spring Harb Symp Quant Biol 64: 517-26, 1999). Members of the NF-AT family share a DNA binding domain of about 300 amino acid residues with approximately 70% sequence similarity. This domain is also termed Rel similarity domain (RSD) due to limited sequence identity to the Rel Homology Domain (RHD) of the NF-κB factors (McCaffrey, P. G. et al., Science 262:750-754, 1993). The RSD domain of NF-AT factors also contains AP-1 interaction motifs and nuclear localization signals. In addition to the RSD domain, NF-AT proteins also have a regulatory domain and at least one N-terminal transactivation domain. The regulatory domain harbors a number of phosphorylation sites.

NF-AT is essential for early T-cell gene activation. NF-AT family members can bind to and transactivate the promoters of multiple cytokine genes including IL-2 and IL-4 (Rooney, J. et al., Immunity 2:545-553, 1995; Szabo, S. J. et al., Mol. Cell. Biol. 13:4793-4805, 1993; Flanagan, W. M. et al., Nature 352:803-807, 1991; and Northrop, J. P. et al., Nature 369:497, 1994). NF-AT appears to be a specific target of immunosuppressants cyclosporin A and FK506 action because transcription directed by this protein is blocked in T cells treated with these drugs, with little or no effect on other transcription factors such as AP-1 and NF-κB.

NF-AT binding sites in cytokine promoter regulatory regions usually are accompanied by nearby sites that bind auxiliary transcription factors, usually members of the AP-1 family. It has been shown that NF-AT and AP-1 proteins bind coordinately and cooperatively and are required for full activity of the IL-2 and IL-4 promoters. Different AP-1 proteins, specifically c-Jun, c-Fos, Fra-1, Fra-2, Jun B and Jun D, have been shown to bind to these sites (Rao et al., Immunol. Today 15:274-281, 1994; Jain et al., Nature 365:352-355, 1993; Boise et al., Mol. Cell. Biol. 13:1911-1919, 1993; Rooney et al., Immunity 2:545-553, 1995; and Rooney et al., Mol. Cell. Biol. 15:6299-6310, 1995).

Modulation of NF-AT bioactivities (e.g., transcription-regulating function) or its cellular level would affect various cellular processes and provide therapeutic means for treating a number of diseases and conditions. There is a need in the art for novel methods and compositions for modulating NF-AT activities and thereby treating diseases or disorders mediated by abnormal activities of lymphocytes (e.g., T cells). The instant invention fulfills this and other needs.

### SUMMARY OF THE INVENTION

In one aspect, the present invention provides methods for identifying an agent that modulates a bioactivity of the NF-AT transcription factor. The methods comprise (a) assaying a biological activity of an NF-AT-modulatory polypeptide of the present invention or its fragment in the presence of a test agent to identify one or more modulating agents that modulate the biological activity of the polypeptide, and (b) testing one or more of the modulating agents for ability to modulate an NF-AT bioactivity.

In some of the methods, the modulating agents are tested for ability to modulate NF-AT in regulating expression of an NF-AT responsive gene. In some other methods, the modulating agents are tested for ability to modulate cellular level of NF-AT. In some of the methods, the NF-AT-modulatory polypeptide is a kinase and the biological activity is phosphorylation of a second polypeptide. In some methods, the NF-AT-modulatory polypeptide is a protease and the biological activity is proteolysis of a second polypeptide. The second polypeptide can be NF-AT or a fragment of NF-AT.

In some of the methods, the test agent is assayed for ability to modulate cellular level of the NF-AT-modulatory polypeptide. The assaying of the biological activity of the NF-AT-modulatory polypeptide can occur in a cell. In some of these methods, the NF-AT-modulatory polypeptide is expressed from the polynucleotide that has been introduced into the cell.

In some methods, the NF-AT bioactivity is inducing expression of a second polynucleotide that is operably linked to an NF-AT response element. The second polynucleotide can encode a reporter polypeptide. In some of these methods, the testing for ability to modulate an NF-AT bioactivity comprises (a) providing a cell or cell lysate that comprises the second polynucleotide that is operably linked to the NF-AT response element, (b) contacting the cell or cell lysate with the test agent, and (c) detecting an increase or decrease in expression of the second polynucleotide in the presence of the test agent compared to expression of the second polynucleotide in the absence of the test agent.

In some methods, the testing for ability to modulate an NF-AT bioactivity comprises contacting a cell or cell lysate with the test agent and determining cellular level of NF-AT or a fragment of NF-AT. In some other methods, the testing for ability to modulate the NF-AT bioactivity comprises contacting a cell or cell lysate with the test agent and determining ability of NF-AT to bind to a second polynucleotide that comprises an NF-AT response element in the cell or cell lysate.

In a related aspect, the present invention provides methods for identifying an agent that modulates cellular level of NF-AT. The methods comprise (a) assaying a biological activity of an NF-AT-modulatory polypeptide of the present invention in the presence of a test agent to identify a modulating agent that modulates the biological activity of the polypeptide, and (b) testing the modulating agent for ability to modulate cellular level of NF-AT. In some of these methods, the NF-AT-modulatory polypeptide is a transcription regulatory protein and the biological activity is transcription of a second polynucleotide. The second polynucleotide encodes an NF-AT or a fragment of the NF-AT. In some other methods, the testing comprises (a) contacting the modulating agent with a second polynucleotide operably linked to a transcription regulatory element of NF-AT, and (b) detecting a change in cellular level of the second polynucleotide relative to cellular level of the second polynucleotide in the absence of the modulating agent. The second polynucleotide can encode a reporter polypeptide. The second polynucleotide can also encode an NF-AT or a fragment of the NF-AT.

In another aspect, the invention provides methods for identifying an agent that modulates expression of an NF-AT responsive gene. These methods comprise (a) contacting a test agent with an NF-AT-modulatory polypeptide of the invention, (b) detecting a change in an activity of the NF-AT-modulatory polypeptide relative to the activity in the absence of the test agent, and (c) detecting a change of expression level of the NF-AT responsive gene in the presence of the test agent identified in (b) relative to expression level of the NF-AT responsive gene in the absence of the test agent.

In still another aspect, the invention provides methods for modulating an NF-AT bioactivity in a cell. The methods comprise administering to the cell an effective amount of an NF-AT modulatory polypeptide of the invention or other NF-AT modulator identified in accordance with the invention. In some of these methods, the NF-AT modulatory polypeptide or its fragment is expressed from an expression vector that has been introduced into the cell. The modulating can be increasing cellular level of NF-AT or decreasing cellular level of NF-AT.

A further understanding of the nature and advantages of the present invention may be realized by reference to the remaining portions of the specification and claims.

### DETAILED DESCRIPTION

The present invention provides novel modulators of the NF-AT transcription factors and methods for identifying novel NF-AT modulators. The invention also provides methods for modulating NF-AT bioactivities in a cell and for treating diseases or conditions mediated by abnormal bioactivities or cellular level of the NF-AT transcription factors. The following sections provide guidance for making and using the compositions of the invention, and for carrying out the methods of the invention.

I. Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). In addition, the following definitions are provided to assist the reader in the practice of the invention.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, or a chemical compound, or a combination of two or more substances. Unless otherwise specified, the terms “agent”, “substance”, and “compound” can be used interchangeably.

The term “analog” is used herein to refer to a molecule that structurally resembles a reference molecule but which has been modified in a targeted and controlled manner, by replacing a specific substituent of the reference molecule with an alternate substituent. Compared to the reference molecule, an analog would be expected, by one skilled in the art, to exhibit the same, similar, or improved utility. Synthesis and screening of analogs, to identify variants of known compounds having improved traits (such as higher binding affinity for a target molecule) is an approach that is well known in pharmaceutical chemistry.

As used herein, “contacting” has its normal meaning and refers to combining two or more agents (e.g., polypeptides or small molecule compounds) or combining agents and cells (e.g., a polypeptide and a cell). Contacting can occur in vitro, e.g., combining two or more agents or combining a test agent and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

A “heterologous sequence” or a “heterologous nucleic acid,” as used herein, is one that originates from a source foreign to the particular host cell, or, if from the same source, is modified from its original form. Thus, a heterologous gene in a host cell includes a gene that, although being endogenous to the particular host cell, has been modified. Modification of the heterologous sequence can occur, e.g., by treating the DNA with a restriction enzyme to generate a DNA fragment that is capable of being operably linked to the promoter. Techniques such as site-directed mutagenesis are also useful for modifying a heterologous nucleic acid.

The term “homologous” when referring to proteins and/or protein sequences indicates that they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTP and BLASTN using default parameters) are described herein.

A “host cell,” as used herein, refers to a prokaryotic or eukaryotic cell to which a heterologous polynucleotide can be introduced. The polynucleotide can be introduced into the cell by any means, e.g., electroporation, calcium phosphate precipitation, microinjection, transformation, viral infection, and/or the like.

The term “identical”, “sequence identical” or “sequence identity” in the context of two nucleic acid sequences or amino acid sequences refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View, Calif.; and GAP, BESTFIT, BLAST, FASTA, or TFASTA in the Wisconsin Genetics Software Package, Genetics Computer Group (GCG), 575 Science Dr., Madison, Wis., U.S.A.). The CLUSTAL program is well described by Higgins and Sharp (1988) Gene 73:237-244; Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment. In some embodiments, the polypeptides herein are at least 70%, generally at least 75%, optionally at least 80%, 85%, 90%, 95% or 99% or more identical to a reference polypeptide (e.g., an NF-AT-modulatory polypeptide encoded by a polynucleotide in Table 1). The percentage can be as measured by, e.g., BLASTP or CLUSTAL or any other available alignment software using default parameters. Similarly, nucleic acids can also be described with reference to a starting nucleic acid, e.g., they can be 50%, 60%, 70%, 75%, 80%, 85%, 90%, 95%, 99% or more identical to a reference nucleic acid, as measured by, e.g., BLASTN, or CLUSTAL, or any other available alignment software using default parameters.

The terms “substantially identical” nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

The term “isolated” means that the material is removed from its original environment (e.g., the natural environment if it is naturally occurring). For example, a naturally occurring nucleic acid, polypeptide, or cell present in a living animal is not isolated, but the same polynucleotide, polypeptide, or cell separated from some or all of the coexisting materials in the natural system, is isolated, even if subsequently reintroduced into the natural system. Such nucleic acids can be part of a vector and/or such nucleic acids or polypeptides could be part of a composition, and still be isolated in that such vector or composition is not part of its natural environment.

The terms “nucleic acid” and “polynucleotide” refer to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. A “polynucleotide sequence” is a nucleic acid (which is a polymer of nucleotides (A, C, T, U, G, etc. or naturally occurring or artificial nucleotide analogues) or a character string representing a nucleic acid, depending on context. Either the given nucleic acid or the complementary nucleic acid can be determined from any specified polynucleotide sequence.

The term “modulate” with respect to NF-AT bioactivities refers to a change in the cellular level or other biological activities of the NF-AT transcription factors. Modulation of NF-AT bioactivities can be up-regulation (i.e., activation or stimulation) or down-regulation (i.e. inhibition or suppression). For example, modulation may cause a change in cellular level of NF-AT, enzymatic modification (e.g., phosphorylation) of NF-AT, binding characteristics (e.g., binding to a target transcription regulatory element), or any other biological, functional, or immunological properties of NF-AT proteins. The change in activity can arise from, for example, an increase or decrease in expression of the NF-AT gene, the stability of mRNA that encodes the NF-AT protein, translation efficiency, or from a change in other bioactivities of the NF-AT transcription factors (e.g., regulating expression of an NF-AT responsive gene). The mode of action of an NF-AT modulator can be direct, e.g., through binding to the NF-AT protein or to genes encoding the NF-AT protein. The change can also be indirect, e.g., through binding to and/or modifying (e.g., enzymatically) another molecule which otherwise modulates NF-AT (e.g., a kinase that specifically phosphorylates NF-AT).

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions which function similarly. Such modified or substituted oligonucleotides are often preferred over native forms because of desirable properties such as, for example, enhanced cellular uptake, enhanced binding to target and increased stability in the presence of nucleases.

The term “operably linked” refers to a functional relationship between two or more polynucleotide (e.g., DNA) segments. Typically, it refers to the functional relationship of a transcriptional regulatory sequence to a transcribed sequence. For example, an NF-AT promoter or enhancer sequence, is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance. A polylinker provides a convenient location for inserting coding sequences so the genes are operably linked to the NF-AT promoter. Polylinkers are polynucleotide sequences that comprise a series of three or more closely spaced restriction endonuclease recognition sequences.

The term “polypeptide” is used interchangeably herein with the terms “polypeptides” and “protein(s)”, and refers to a polymer of amino acid residues, e.g., as typically found in proteins in nature. A “mature protein” is a protein which is full-length and which, optionally, includes glycosylation or other modifications typical for the protein in a given cell membrane.

The promoter region of a gene includes the transcription regulatory elements that typically lie 5′ to a structural gene. If a gene is to be activated, proteins known as transcription factors attach to the promoter region of the gene. This assembly resembles an “on switch” by enabling an enzyme to transcribe a second genetic segment from DNA into RNA. In most cases the resulting RNA molecule serves as a template for synthesis of a specific protein; sometimes RNA itself is the final product. The promoter region may be a normal cellular promoter or an oncopromoter.

The term “recombinant” has the usual meaning in the art, and refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. When used with reference to a cell, the term indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “recombinant expression cassette” or simply an “expression cassette” is a nucleic acid construct, generated recombinantly or synthetically, that has control elements that are capable of affecting expression of a structural gene that is operably linked to the control elements in hosts compatible with such sequences. Expression cassettes include at least promoters and optionally, transcription termination signals. Typically, the recombinant expression cassette includes at least a nucleic acid to be transcribed and a promoter. Additional factors necessary or helpful in effecting expression can also be used as described herein. For example, transcription termination signals, enhancers, and other nucleic acid sequences that influence gene expression, can also be included in an expression cassette.

Transcription refers to the process involving the interaction of an RNA polymerase with a gene, which directs the expression as RNA of the structural information present in the coding sequences of the gene. The process includes, but is not limited to the following steps: (1) transcription initiation, (2) transcript elongation, (3) transcript splicing, (4) transcript capping, (5) transcript termination, (6) transcript polyadenylation, (7) nuclear export of the transcript, (8) transcript editing, and (9) stabilizing the transcript.

A transcription regulatory element or sequence include, but is not limited to, a promoter sequence (e.g., the TATA box), an enhancer element, a signal sequence, or an array of transcription factor binding sites. It controls or regulates transcription of a gene operably linked to it.

A “variant” of a molecule such as a modulator of NF-AT is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical.

A “vector” is a composition for facilitating introduction, replication and/or expression of a selected nucleic acid in a cell. Vectors include, e.g., plasmids, cosmids, viruses, YACs, bacteria, poly-lysine, etc. A “vector nucleic acid” is a nucleic acid molecule into which heterologous nucleic acid is optionally inserted which can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (primarily in yeast and bacteria) “artificial chromosomes.” “Expression vectors” are vectors that comprise elements that provide for or facilitate transcription of nucleic acids that are cloned into the vectors. Such elements can include, e.g., promoters and/or enhancers operably coupled to a nucleic acid of interest.

II. Identification of Novel NF-AT-Modulatory Polypeptides

Human NF-AT gene encodes a 393 amino acid residue, 53 kD phospho-protein. The protein is divided structurally and functionally into four domains. The first 42 amino acids at the N-terminus constitute a transcriptional activation machinery in positively regulating gene expression. Amino acid residues 13-23 in the NF-AT protein are identical in a number of diverse species and certain amino acids in this region have been shown to be required for transcriptional activation by the protein in vivo. The sequence-specific DNA binding domain of NF-AT is localized between amino acid residues 102 and 292. The native NF-AT is a tetramer in solution, and amino acid residues 324-355 are required for this oligomerization of the protein. The C-terminal 26 amino acids form an open domain composed of nine basic amino acid residues that bind to DNA and RNA readily with some sequence or structural preferences. There is evidence that the NF-AT protein requires a structural change to activate it for sequence specific binding to DNA. Deletion of the C-terminus domain activates site-specific DNA binding by the central domain.

As used in the present invention, the consensus binding sites on a target gene that is regulated by the NF-AT transcription factors (i.e., an NF-AT responsive gene) are interchangeably termed “NF-AT recognition sequences,” “NF-AT response elements,” or “NF-AT binding sites.” These sequences are found in many NF-AT responsive genes and usually have a consensus NF-AT DNA binding motif, e.g., GGAAA or ACAGGAAGT (Rivera et al., J Biol Chem 273: 22382-8, 1998; and Koizumi et al., Mol Cell Biol 13: 6690-701, 1993). As detailed below, NF-AT binding sites have been found in a great number of genes (especially genes encoding lymphokines), e.g., IL-4 (Li-Weber et al., Gene 188: 253-60, 1997), IL-5 (Boer et al., Int. J. Biochem. Cell Biol. 31: 1221-36, 1999), Fas ligand (Dzialo-Hatton et al., J Immunol 166: 4534-42, 2001), and IL-2 (Hivroz-Burgaud et al., Eur J Immunol 21: 2811-9, 1991).

The present invention provides novel protein or polypeptide modulators that modulate NF-AT. Utilizing an expression vector which expresses a reporter gene under the control of an NF-AT responsive sequence (Fiering et al., Genes Devel. 4:1823-1834, 1990), a number of polynucleotides were identified which up-regulate expression of the reporter gene when the expression vector and the polynucleotides were co-transfected into a host cell (see Examples below). Table 1 lists exemplary polynucleotides encoding such NF-AT-modulatory polypeptides. As shown in the Table, the novel NF-AT-modulatory polypeptides include very diversified classes of proteins, including kinases, protease inhibitors, DNA-binding proteins, RNA binding proteins, receptor polypeptides, and etc.

The NF-AT-modulatory polypeptides identified by the present inventors can operate with a number of mechanisms in modulating NF-AT. For example, they can modulate upstream pathways leading to NF-AT activation (e.g., a kinase pathway). Activation of the NF-AT pathway requires activation of the T cell receptor (TCR) and stimulation of several protein tyrosine kinases. TCR activation leads to a rise in intracellular calcium concentration and calcineurin activation, the latter mediating nuclear translocation of NF-AT factors (see, e.g., Liu, Immunol. Today 14: 290-5, 1993; and Crabtree, Cell 96: 611-4, 1999). Stimulation of the protein tyrosine kinases activates a number of Ser/Thr protein kinases which in turn controls transcriptional activation of NF-ATs and induction of AP-1 (see, e.g., Avots et al., Immunity 10: 515-24, 1999; Treisman, Curr. Opin. Cell Biol. 8: 205-15, 1996; and Serfling et al., Biochim Biophys Acta 1498: 1-18, 2000).

Further, the modulation could be the result of altered activities of endogenous NF-AT that in turn modulates expression of the reporter gene. For example, the NF-AT-modulatory polypeptides of the present invention could exert regulatory function on expression of the NF-AT gene and cellular level of the NF-AT protein. They can stimulate or inhibit expression of the NF-AT gene or otherwise alter cellular level of the NF-AT protein by, e.g., modulating events relating to transcription of the NF-AT gene, modulating post-transcriptional processing, modulating translation of NF-AT, modulating post-translational modification, or modulating stability or proteolysis of the NF-AT protein.

Other than modulating cellular level of endogenous NF-AT, the NF-AT-modulatory polypeptides can also act by modulating other biological activities that are necessary for or involved in the transcription-regulating function of NF-ATs. For example, they can modulate phosphorylation of the NF-AT protein. Phosphorylation of NF-AT plays an important role in the transcription-regulating function of NF-ATs, e.g., DNA-binding activities (Park et al., Blood 82: 2470-7, 1993; and Behrens et al., Proc Natl Acad Sci USA 98: 1769-74, 2001). A number of protein kinases are known to phosphorylate NF-ATs, e.g., GSK3, CKI, CKII, and JNK (Beals et al., Science 275: 1930-34, 1997; Porter et al., J. Biol. Chem. 275: 3542-51, 2000; Chow et al., Science 278: 1638-41; and Serfling et al., Biochim Biophys Acta 1498: 1-18, 2000). Dephosphorylation of NF-AT is mediated by protein phosphatases such as calcineurin (Rao et al., Annu. Rev. Immunol. 15: 707-47, 1997). Calcineurin interacts directly with several motifs in the regulatory domain of NA-AT proteins (Luo et al., Proc. Natl. Acad. Sci USA 93: 4755-61, 1996; and Masuda et al., Mol. Cell. Biol. 17: 2066-75, 1997).

The NF-AT-modulatory polypeptides can also modulate NF-AT interaction with other transcription factors or proteins that are involved in transcription regulation of NF-AT responsive genes. A number of proteins are known to bind to NF-AT and modulate NF-AT activities. For example, as noted above, the RSD domain of NF-AT contains AP-1 interaction site. It has been shown that NF-AT and AP-1 proteins bind coordinately and cooperatively and are required for full activity of the IL-2 and IL-4 promoters (Rooney et al., Immunity 2: 545-553, 1995; and Rooney et al., Mol. Cell. Biol. 15: 6299-6310, 1995). NF-AT3 also interacts with the cardiac zinc finger transcription factor GATA4, resulting in synergistic activation of cardiac transcription (Molkentin et al., Cell 93: 215-28, 1998). NF-AT-modulatory polypeptides of the present invention could modulate NF-AT interaction with AP-1 or GATA4 in regulating expression of NF-AT responsive genes. The NF-AT-modulatory polypeptides can also modulate NF-AT cellular activities by indirectly modulate any of the proteins or factors that interact with NF-AT (e.g., an AP-1 protein).

TABLE 1
Polynucleotides encoding NF-AT-modulatory polypeptides

| | Fold of | GenBank | Description of the polynucleotide sequence and |
|---|---|---|---|
| | Induction | Acc. No. | encoded polypeptide |
| 1 | 5.15 | BC010760 | Mus musculus, Similar to mannose binding lectin, serum |
| | | | (C), clone MGC: 18500 IMAGE: 4212216, mRNA, complete |
| | | | cds |
| 2 | 4.75 | BC016506 | Mus musculus, guanine nucleotide binding protein (G |
| | | | protein), gamma 4 subunit, clone MGC: 25282 |
| | | | IMAGE: 4502719, mRNA, complete cds. |
| 3 | 5.2 | BC014723 | Mus musculus, Similar to phosphodiesterase 6G, cGMP- |
| | | | specific, rod, gamma, clone MGC: 25416 IMAGE: 4511855, |
| | | | mRNA, complete cds |
| 4 | 5.4 | BC019387 | Mus musculus, clone MGC: 25819 IMAGE: 4164847, |
| | | | mRNA, complete cds. |
| 5 | 7.58 | BC014694 | Mus musculus, Similar to Purkinje cell protein 2 (L7), clone |
| | | | MGC: 25385 IMAGE: 4527572, mRNA, complete cds. |
| 6 | 5.17 | BC014718 | Mus musculus, Similar to deoxyribonuclease I, clone |
| | | | MGC: 25273 IMAGE: 4925690, mRNA, complete cds |
| 7 | 5.81 | BC016101 | Mus musculus, Similar to epithelial apical membrane |
| | | | calcium transporter/channel CaT1 (also termed TRPV6), |
| | | | clone MGC: 27673 IMAGE: 4911355, mRNA, complete cds. |
| 8 | 12.71 | BC022601 | Mus musculus, RIKEN cDNA 4432411H13 gene, clone |
| | | | MGC: 31106 IMAGE: 4160199, mRNA, complete cds |
| 9 | 8.51 | BC003443 | Mus musculus, clone MGC: 6865 IMAGE: 2651122, mRNA, |
| | | | complete cds |
| 10 | 5.41 | BC025572 | Mus musculus, clone MGC: 36598 IMAGE: 5323819, |
| | | | mRNA, complete cds |
| 11 | 9.35 | BC009093 | Mus musculus, early growth response 2, clone MGC: 7113 |
| | | | IMAGE: 3157863, mRNA, complete cds. |
| 12 | 6.61 | BC003282 | Mus musculus, ring finger protein 4, clone MGC: 6684 |
| | | | IMAGE: 3582133, mRNA, complete cds. |
| 13 | 7.13 | BC004699 | Mus musculus, guanine nucleotide regulatory protein |
| | | | (oncogene), clone MGC: 5716 IMAGE: 3499258, mRNA, |
| | | | complete cds. |
| 14 | 40.26 | BC004685 | Mus musculus, clone MGC: 7852 IMAGE: 3501062, mRNA, |
| | | | complete cds |
| 15 | 7.26 | BC004711 | Mus musculus, clone MGC: 7809 IMAGE: 3499974, mRNA, |
| | | | complete cds |
| 16 | 6 | BC010224 | Mus musculus, clone MGC: 6382 IMAGE: 3500685, mRNA, |
| | | | complete cds |
| 17 | 5.13 | BC006690 | Mus musculus, Similar to D-aspartate oxidase, clone |
| | | | MGC: 6692 IMAGE: 3582980, mRNA, complete cds. |
| 18 | 7.63 | BC003244 | Mus musculus, Similar to nucleolar phosphoprotein p130, |
| | | | clone MGC: 6662 IMAGE: 3498349, mRNA, complete cds. |
| 19 | 4.82 | BC004715 | Mus musculus, Similar to silica-induced gene 81, clone |
| | | | MGC: 6048 IMAGE: 3582142, mRNA, complete cds |
| 20 | 5.28 | BC010564 | Mus musculus, H2A histone family, member O, clone |
| | | | MGC: 5956 IMAGE: 3582122, mRNA, complete cds. |
| 21 | 6.79 | BC012255 | Mus musculus, Similar to ubiquitin carrier protein, clone |
| | | | MGC: 6682 IMAGE: 3581845, mRNA, complete cds. |
| 22 | 7.81 | BC004703 | Mus musculus, phenylalkylamine Ca2+ antagonist |
| | | | (emopamil) binding protein, clone MGC: 7785 |
| | | | IMAGE: 3499265, mRNA, complete cds. |
| 23 | 6.15 | BC006680 | Mus musculus, Similar to ubiquitin C, clone MGC: 7811 |
| | | | IMAGE: 3500023, mRNA, complete cds |
| 24 | 7.96 | BC004674 | Mus musculus, Similar to RNA binding motif protein 10, |
| | | | clone MGC: 7826 IMAGE: 3500403, mRNA, complete cds. |
| 25 | 8.89 | BC014772 | Mus musculus, ubiquitin A-52 residue ribosomal protein |
| | | | fusion product 1, clone MGC: 6675 IMAGE: 3500484, |
| | | | mRNA, complete cds. |
| 26 | 5.55 | BC006666 | Mus musculus, clone MGC: 7770 IMAGE: 3499059, mRNA, |
| | | | complete cds |
| 27 | 5.65 | BC008126 | Mus musculus, Similar to pyruvate dehydrogenase kinase, |
| | | | isoenzyme 3, clone MGC: 6383 IMAGE: 3500763, mRNA, |
| | | | complete cds. |
| 28 | 18.25 | BC008573 | Homo sapiens, clone MGC: 17005 IMAGE: 4182067, |
| | | | mRNA, complete cds. |
| 29 | 5.59 | BC017556 | Homo sapiens, likely ortholog of mouse coiled coil forming |
| | | | protein 1, clone MGC: 9519 IMAGE: 3908134, mRNA, |
| | | | complete cds. |
| 30 | 5.18 | BC010541 | Homo sapiens, Similar to RIKEN cDNA 2300002L21 gene, |
| | | | clone MGC: 17528 IMAGE: 3458906, mRNA, complete cds. |
| 31 | 5.83 | BC012389 | Homo sapiens, Similar to transmembrane 4 superfamily |
| | | | member 6, clone MGC: 9097 IMAGE: 3857537, mRNA, |
| | | | complete cds. |
| 32 | 9.82 | BC006825 | Homo sapiens, RNA binding motif protein 3, clone |
| | | | MGC: 5289 IMAGE: 3449185, mRNA, complete cds. |
| 33 | 5.3 | BC000793 | Homo sapiens, eukaryotic translation initiation factor 1A, |
| | | | clone MGC: 5131 IMAGE: 3451631, mRNA, complete cds. |
| 34 | 12.65 | BC000876 | Homo sapiens, Similar to zinc finger protein 174, clone |
| | | | MGC: 5061 IMAGE: 3461658, mRNA, complete cds. |
| 35 | 7.33 | BC025790 | Homo sapiens, serine protease inhibitor, Kazal type 1, clone |
| | | | MGC: 34543 IMAGE: 5225693, mRNA, complete cds. |
| 36 | 6.93 | BC025717 | Homo sapiens, chemokine (C-C motif) receptor-like 2, |
| | | | clone MGC: 34104 IMAGE: 5228561, mRNA, complete cds |
| 37 | 4.94 | BC025791 | Homo sapiens, ghrelin precursor, clone MGC: 39929 |
| | | | IMAGE: 5212768, mRNA, complete cds. |
| 38 | 6.62 | BC012866 | Homo sapiens, Similar to tumor necrosis factor receptor |
| | | | superfamily, member 10a, clone MGC: 9365 |
| | | | IMAGE: 3857315, mRNA, complete cds. |
| 39 | 5.61 | BC011118 | Mus musculus, Similar to CCAAT/enhancer binding protein |
| | | | (C/EBP), alpha, clone MGC: 18705 IMAGE: 4194023, |
| | | | mRNA, complete cds |
| 40 | 4.75 | BC018130 | Homo sapiens, coagulation factor II (thrombin) receptor- |
| | | | like 1, clone MGC: 9298 IMAGE: 3895653, mRNA, |
| | | | complete cds. |
| 41 | 8.82 | BC011965 | Homo sapiens,D site of albumin promoter (albumin D-box) |
| | | | binding protein, clone MGC: 9164 IMAGE: 3857434, |
| | | | mRNA, complete cds. |
| 42 | 8.06 | XM113489 | Homo sapiens similar to FK506-binding protein like; |
| | | | similar to immunophilin-like protein NG7(LOC202518), |
| | | | mRNA |
| 43 | 5.53 | BC012868 | Homo sapiens, serine protease inhibitor, Kunitz type, 2, |
| | | | clone MGC: 9154 IMAGE: 3857277, mRNA, complete cds. |

III. Methods for Screening Modulators of NF-AT

The NF-AT-modulatory polypeptides described above provide novel targets for screening modulators (agonists or antagonists) of the NF-AT transcription factors. The novel NF-AT modulators can be used to modulate transcriptional regulation of NF-AT responsive genes by NF-ATs. The expression of an NF-AT responsive gene can be positively or negatively regulated to provide, respectively, for increased or decreased production of the protein whose expression is mediated by an NF-AT response element. Furthermore, genes that do not have NF-AT response elements in their wild type form can be placed under the control of NF-AT by inserting an NF-AT binding site in an appropriate position, using techniques known to those skilled in the art. Thus, expression of these genes can also be modulated by the NF-AT modulators of the present invention.

A. General Scheme and Assay Systems

Employing the novel NF-AT-modulatory polypeptides described above, the present invention provides methods for screening agents or compounds that modulate activities of the NF-AT transcription factors. Various biochemical and molecular biology techniques well known in the art can be employed to practice the present invention. Such techniques are described in, e.g., Sambrook et al., Molecular Cloning: A Laboratory Manual, Cold Spring Harbor Press, N.Y., Second (1989) and Third (2000) Editions; and Ausubel et al., Current Protocols in Molecular Biology, John Wiley & Sons, Inc., New York (1987-1999).

In some methods, test agents are first assayed for their ability to modulate a biological activity of an NF-AT-modulatory polypeptide (“the first assay step”). Modulating agents thus identified are then subject to further screening for ability to modulate an activity of the NF-AT transcription factors, typically in the presence of the NF-AT-modulatory polypeptide (“the second testing step”). Depending on the NF-AT-modulatory polypeptide employed in the method, modulation of different biological activities of the NF-AT-modulatory polypeptide can be assayed in the first step. For example, a test agent can be assayed for binding to the NF-AT-modulatory polypeptide. The test agent can be assayed for activity to modulate expression level of the NF-AT-modulatory polypeptide, e.g., transcription or translation. The test agent can also be assayed for activities in modulating cellular level or stability of the NF-AT-modulatory polypeptide, e.g., post-translational modification or proteolysis.

If the NF-AT-modulatory polypeptide has a known or well-established biological or enzymatic function (e.g., kinase activity, protease activity, or DNA-binding activity), the biological activity monitored in the first screening step can be the specific biochemical or enzymatic activity of the NF-AT-modulatory polypeptide. In an exemplary embodiment, the NF-AT-modulatory polypeptide is a kinase (e.g., encoded by BC008126), and test agents are first screened for modulating the kinase's activity in phosphorylating a substrate. The substrate can be a polypeptide known to be phosphorylated by the kinase. The substrate can also be an NF-AT transcription factor or an NF-AT fragment harboring the kinase binding site and the phosphorylation site (e.g., a functional derivative of an NF-AT transcription factor).

Once test agents that modulate the NF-AT-modulatory polypeptides are identified, they are typically further tested for ability to modulate the NF-AT transcription factors. The test agents can be further tested for ability to modulate expression or cellular level of NF-AT or fragment thereof. Alternatively, the test agents can be further tested for activity in modulating transcription-regulating function of NF-AT, e.g., binding to an NF-AT recognition sequence or promoting expression of a gene under the control of an NF-AT binding sequence (i.e., an NF-AT responsive gene).

As noted above, the NF-AT-modulatory polypeptides identified by the present inventors can modulate cellular level of NF-AT or transcription-regulating functions of NF-AT. If a test agent identified in the first screening step modulates cellular level (e.g., by altering transcription activity) of the NF-AT-modulatory polypeptide, it would indirectly modulate the NF-AT transcription factors. For example, if the NF-AT-modulatory polypeptide (e.g., a kinase) modulates NF-AT activities by specifically phosphorylating NF-AT, a test agent which alters cellular level of the NF-AT-modulatory kinase would indirectly also modulate NF-AT activities. Similarly, if the NF-AT-modulatory polypeptide modulates cellular level of NF-AT, a test agent that modulates cellular level of the NF-AT-modulatory polypeptide would indirectly alter cellular level of NF-AT.

On the other hand, if a test agent modulates an activity other than cellular level of the NF-AT-modulatory polypeptide, then the further testing step is needed to confirm that their modulatory effect on the NF-AT-modulatory polypeptide will indeed lead to modulation of NF-AT activities (e.g., cellular level of NF-ATs or transcription-regulating function of NF-ATs). For example, a test agent that modulates phosphorylation activity of an NF-AT-modulatory polypeptide needs to be further tested in order to confirm that modulation of phosphorylation activity of the NF-AT-modulatory polypeptide can result in modulation of transcription-regulating function or cellular level of NF-ATs.

In both the first assaying step and the second testing step, either an intact NF-AT-modulatory polypeptide and an NF-AT transcription factor, or their fragments, analogs, or functional derivatives can be used. The fragments that can be employed in these assays usually retain one or more of the biological activities of the NF-AT-modulatory polypeptide (e.g., kinase activity if the NF-AT-modulatory employed in the first assaying step is a kinase) and the NF-AT transcription factor (e.g., binding to an NF-AT recognition sequence). Fusion proteins containing such fragments or analogs can also be used for the screening of test agents. Functional derivatives of NF-AT-modulatory polypeptides and NF-ATs usually have amino acid deletions and/or insertions and/or substitutions while maintaining one or more of the bioactivities and therefore can also be used in practicing the screening methods of the present invention. A functional derivative of an NF-AT-modulatory polypeptide or an NF-AT transcription factor can be prepared from a naturally occurring or recombinantly expressed protein by proteolytic cleavage followed by conventional purification procedures known to those skilled in the art. Alternatively, the functional derivative can be produced by recombinant DNA technology by expressing only fragments of an NF-AT-modulatory polypeptide or NF-AT that retains one or more of their bioactivities.

A variety of routinely practiced assays can be used to identify test agents that modulate an NF-AT-modulatory polypeptide or NF-AT. Preferably, the test agents are screened with a cell based assay system. For example, in a typical cell based assay for screening NF-AT modulators (i.e., the second screening step), a construct comprising an NF-AT transcription regulatory element operably linked to a reporter gene is introduced into a host cell system. The reporter gene activity (e.g., an enzymatic activity) in the presence of a test agent can be determined and compared to the activity of the reporter gene in the absence of the test agent. An increase or decrease in the activity identifies a modulator of the NF-AT transcription factor. The reporter gene can encode any detectable polypeptide (response or reporter polypeptide) known in the art, e.g., detectable by fluorescence or phosphorescence or by an enzymatic activity. The detectable response polypeptide can be, e.g., luciferase, alpha-glucuronidase, alpha-galactosidase, chloramphenicol acetyl transferase, green fluorescent protein, enhanced green fluorescent protein, and the human secreted alkaline phosphatase.

In the cell-based assays, the test agent (e.g., a peptide or a polypeptide) can also be expressed from a different vector that is also present in the host cell. In some methods, a library of test agents are encoded by a library of such vectors (e.g., a cDNA library as employed in the Examples below). Such libraries can be generated using methods well known in the art (see, e.g., Sambrook et al. and Ausubel et al., supra) or obtained from a variety of commercial sources.

In addition to cell-based assays described above, modulators of NF-ATs can also be screened with non-cell based methods. These methods include, e.g., mobility shift DNA-binding assays, methylation and uracil interference assays, DNase and hydroxy radical footprinting analysis, fluorescence polarization, and UV crosslinking or chemical cross-linkers. For a general overview, see, e.g., Ausubel et al., supra (chapter 12, DNA-Protein Interactions). One technique for isolating co-associating proteins, including nucleic acid and DNA/RNA binding proteins, includes use of UV crosslinking or chemical cross-linkers, including e.g., cleavable cross-linkers dithiobis (succinimidylpropionate) and 3,3′-dithiobis (sulfosuccinimidyl-propionate); see, e.g., McLaughlin (1996) Am. J. Hum. Genet. 59:561-569; Tang (1996) Biochemistry 35:8216-8225; Lingner (1996) Proc. Natl. Acad. Sci. USA 93:10712; Chodosh (1986) Mol. Cell. Biol 6:4723-4733.

B. Test Agents

Test agents that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins.

The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small organic molecules (e.g., molecules with a molecular weight of not more than about 1,000). Preferably, high throughput assays are adapted and used to screen for such small molecules. In some methods, combinatorial libraries of small molecule test agents as described above can be readily employed to screen for small molecule modulators of NF-ATs. A number of assays are available for such screening, e.g., as described in Schultz (1998) Bioorg Med Chem Lett 8:2409-2414; Weller (1997) Mol Divers. 3:61-70; Fernandes (1998) Curr Opin Chem Biol 2:597-603; and Sittampalam (1997) Curr Opin Chem Biol 1:384-91.

Libraries of test agents to be screened with the claimed methods can also be generated based on structural studies of the NF-AT-modulatory polypeptides, their fragments or analogs. Such structural studies allow the identification of test agents that are more likely to bind to the NF-AT-modulatory polypeptides. The three-dimensional structure of an NF-AT-modulatory polypeptide can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of NF-AT-modulatory polypeptides' structures provides another means for designing test agents for screening NF-AT modulators. Methods of molecular modeling have been described in the literature, e.g., U.S. Pat. No. 5,612,894 entitled “System and method for molecular modeling utilizing a sensitivity factor,” and U.S. Pat. No. 5,583,973 entitled “Molecular modeling method and system.” In addition, protein structures can also be determined by neutron diffraction and nuclear magnetic resonance (NMR). See, e.g., Physical Chemistry, 4th Ed. Moore, W. J. (Prentice-Hall, New Jersey 1972), and NMR of Proteins and Nucleic Acids, K. Wuthrich (Wiley-Interscience, New York 1986).

Modulators of the present invention also include antibodies that specifically bind to an NF-AT-modulatory polypeptide in Table 1. Such antibodies can be monoclonal or polyclonal. Such antibodies can be generated using methods well known in the art. For example, the production of non-human monoclonal antibodies, e.g., murine or rat, can be accomplished by, for example, immunizing the animal with an NF-AT-modulatory polypeptide or its fragment (See Harlow & Lane, Antibodies, A Laboratory Manual, Cold Spring Harbor Laboratory Press, Cold Spring Harbor N.Y.). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to an NF-AT-modulatory polypeptide of the present invention.

Human antibodies against an NF-AT-modulatory polypeptide can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an NF-AT-modulatory polypeptide or its fragment.

C. Screening Test Agents that Modulate NF-AT-Modulatory Polypeptides

A number of assay systems can be employed to screen test agents for modulators of an NF-AT-modulatory polypeptide. As noted above, the screening can utilize an in vitro assay system or a cell-based assay system. In this screening step, test agents can be screened for binding to the NF-AT-modulatory polypeptide, altering cellular level of the NF-AT-modulatory polypeptide, or modulating other biological activities of the NF-AT-modulatory polypeptide.

1. Binding of Test Agents to an NF-AT-Modulatory Polypeptide

In some methods, binding of a test agent to an NF-AT-modulatory polypeptide is determined in the first screening step. Binding of test agents to an NF-AT-modulatory polypeptide can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the NF-AT-modulatory polypeptide, e.g., co-immunoprecipitation with the NF-AT-modulatory polypeptide by an antibody directed to the NF-AT-modulatory polypeptide. The test agent can also be identified by detecting a signal that indicates that the agent binds to the NF-AT-modulatory polypeptide, e.g., fluorescence quenching.

Competition assays provide a suitable format for identifying test agents that specifically bind to an NF-AT-modulatory polypeptide. In such formats, test agents are screened in competition with a compound already known to bind to the NF-AT-modulatory polypeptide. The known binding compound can be a synthetic compound. It can also be an antibody, which specifically recognizes the NF-AT-modulatory polypeptide, e.g., a monoclonal antibody directed against the NF-AT-modulatory polypeptide. If the test agent inhibits binding of the compound known to bind the NF-AT-modulatory polypeptide, then the test agent likely also binds the NF-AT-modulatory polypeptide.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, “Antibodies, A Laboratory Manual,” Cold Spring Harbor Press (1988)); solid phase direct label RIA using ¹²⁵I label (see Morel et al., Mol. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an NF-AT-modulatory polypeptide or its fragments onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the NF-AT-modulatory polypeptide, the test agents are bound to the solid matrix and the NF-AT-modulatory polypeptide molecule is then added.

Soluble assays include some of the combinatory libraries screening methods described above. Under the soluble assay formats, neither the test agents nor the NF-AT-modulatory polypeptide are bound to a solid support. Binding of an NF-AT-modulatory polypeptide or fragment thereof to a test agent can be determined by, e.g., changes in fluorescence of either the NF-AT-modulatory polypeptide or the test agents, or both. Fluorescence may be intrinsic or conferred by labeling either component with a fluorophor.

In some binding assays, either the NF-AT-modulatory polypeptide, the test agent, or a third molecule (e.g., an antibody against the NF-AT-modulatory polypeptide) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

2. Agents Modulating Other Activities of NF-AT-Modulatory Polypeptides

Binding of a test agent to an NF-AT-modulatory polypeptide provides an indication that the agent can be a modulator of the NF-AT-modulatory polypeptide. It also suggests that the agent may modulate NF-AT bioactivities (e.g., by binding to and modulate the NF-AT-modulatory polypeptide which in turn acts on an NF-AT transcription factor). Thus, a test agent that binds to an NF-AT-modulatory polypeptide can be further tested for ability to modulate NF-AT activities (i.e., in the second testing step outlined above). Alternatively, a test agent that binds to an NF-AT-modulatory polypeptide can be further examined to determine its activity on the NF-AT-modulatory polypeptide. The existence, nature, and extent of such activity can be tested by an activity assay. Such an activity assay can confirm that the test agent binding to the NF-AT-modulatory polypeptide indeed has a modulatory activity on the NF-AT-modulatory polypeptide.

More often, activity assays can be used independently to identify test agents that modulate activities of an NF-AT-modulatory polypeptide (i.e., without first assaying their ability to bind to the NF-AT-modulatory polypeptide). In general, such methods involve adding a test agent to a sample containing an NF-AT-modulatory polypeptide in the presence of other molecules or reagents which are necessary to test a biological activity of the NF-AT-modulatory polypeptide (e.g., kinase activity if the NF-AT-modulatory polypeptide is a kinase), and determining an alteration in the biological activity of the NF-AT-modulatory polypeptide. In addition to assays for screening agents that modulate an enzymatic or other biological activities of an NF-AT-modulatory polypeptide, the activity assays also encompass in vitro screening and in vivo screening for alterations in expression or cellular level of the NF-AT-modulatory polypeptide.

In an exemplary embodiment, the NF-AT-modulatory polypeptide is a kinase, and the test agent is examined for ability to modulate the kinase activity of the NF-AT-modulatory polypeptide. Methods for monitoring kinase activity and NF-AT phosphorylation are described, e.g., in Beals et al., Science 275: 1930-34, 1997; Porter et al., J. Biol. Chem. 275: 3542-51, 2000; and Chow et al., Science 278: 1638-41. Any of these methods can be employed to assay modulatory effect of a test agent on an NF-AT-modulatory polypeptide (e.g., one encoded by GenBank Acc. No. BC008126).

D. Screening agents that modulate NF-ATs

Once a modulating agent has been identified to bind to an NF-AT-modulatory polypeptide and/or to modulate a biological activity (including cellular level) of the NF-AT-modulatory polypeptide, it can be further tested for ability to modulate bioactivities of the NF-AT transcription factors. Modulation of NF-AT bioactivities by the modulating agent is typically tested in the presence of the NF-AT-modulatory polypeptide. When a cell-based screening system is employed, the NF-AT-modulatory polypeptide can be expressed from an expression vector that has been introduced into a host cell. The NF-AT transcription factor or an NF-AT fragment can be expressed from a second expression vector. Alternatively, the NF-AT transcription factor can be supplied endogenously by the host cell in the screening system.

1. NF-AT Bioactivities to be Monitored

Unless otherwise specified, modulation of bioactivities of the NF-AT transcription factors includes modulation of cellular level of NF-AT, as well as other biological or cellular activities of the NF-AT transcription factors. The term “NF-AT bioactivity” or “biological activity of NF-AT” include biochemical properties of NF-ATs and physiological roles played by the NF-AT transcription factors in regulating cellular processes. The NF-AT transcription factor is involved in a very broad range of biological pathways and cellular activities (see, e.g., Serfling et al., Biochim Biophys Acta, 1498:1-18, 2000). The broad spectrum of NF-AT bioactivities has been disclosed in the literature and in the present invention (e.g., Section II above and references cited therein). For example, activation of the NF-AT pathway leads to trans-regulation of expression of numerous target genes (NF-AT responsive genes) such as lymphokine genes. NF-ATs play important roles in T cell differentiation by inducing expression of the various lymphokine genes. NF-ATs participate in control of the cell cycle and in the control of the generation of Th1 and Th2 effector cells. NF-ATs may also be important factors in controlling apoptosis and cancerigenesis of T cells.

Thus, NF-AT bioactivities to be monitored in this screening step include, but are not limited to, transcription or translation of NF-ATs, cellular level of NF-ATs, enzymatic or non-enzymatic modification (e.g., phosphorylation) of NF-AT, binding characteristics (e.g., binding to a target transcription regulatory element), regulation of expression of NF-AT responsive genes, interaction with another regulatory protein or molecule (e.g., AP-1), and regulation of T cell proliferation or lymphokine production. All these bioactivities can be tested in the presence of a modulating agent that has been identified to bind to and/or modulate an NF-AT-modulatory polypeptide.

2. Screening for NF-AT Modulators

Modulation of cellular level or other bioactivities of the NF-AT transcription factors can be determined in a non-cell based assay system or cell-based assays, similar to the first screening step for identifying modulators of NF-AT-modulatory polypeptides. Using eukaryotic in vitro transcription systems, effects of test agents on cellular level or activities of an NF-AT can be tested by directly measuring in the presence of the test agents expression or cellular level of the NF-AT, or its transcription-regulating activity. Because the test agent is likely to exert its modulatory effect on the NF-AT by modulating an NF-AT-modulatory polypeptide, the NF-AT-modulatory polypeptide is typically also present in the assay system.

With cell-based assays, vectors expressing a reporter gene or other linked polynucleotides under the control of a transcription regulatory element of an NF-AT gene (for assaying modulation of NF-AT expression) or an NF-AT recognition sequence (for assaying modulation of NF-AT transcription-regulating activities) are introduced into appropriate host cells. Modulation of NF-AT activities are typically examined by measuring expression of the reporter genes or other linked polynucleotides. An altered activity of the reporter gene (e.g., its expression level) in the presence of a test agent would indicate that the test agent is a modulator of the NF-AT.

If an NF-AT recognition sequence is used in the expression vector, an observed modulation of the reporter gene could be due to a direct interaction between the test agent with the expression vector. The modulation could also be due to an altered activity of endogenous NF-AT (e.g., its DNA-binding activity or cellular level) as a result of the presence of the test agent. The test agent's activity on the endogenous NF-AT could be direct, e.g., by interacting directly with NF-AT, or indirect, e.g., through interacting with another molecule (e.g., an NF-AT modulatory polypeptide) that in turn binds to the NF-AT polypeptide. If the test agent was first identified to modulate an NF-AT-modulatory polypeptide in the first screening step, its modulation on NF-AT activities or cellular level is likely to be indirect (i.e., through its interaction with the NF-AT-modulatory polypeptide).

Various assays for analyzing NF-AT bioactivities have been described in the art and can be readily employed to screen for test agents that modulate NF-AT activities. For example, expression of NF-AT or cellular levels of NF-AT can be measured using routinely practiced methods (e.g., Sambrook et al., supra; and Ausubel et al., supra), as well as numerous methods described in the literatures (e.g., Hivroz-Burgaud et al., Eur J Immunol 21: 2811-9, 1991; Baldari et al., Biologicals 26: 1-5, 1998; Han et al., Toxicol Lett 108: 1-10, 1999; and Akioka et al., Transplant Proc 31: 2745-6, 1999). Modulation of various other biological activities of NF-ATs by a test agent can also be assayed in accordance with many methods that have been disclosed in the art, e.g., Suzuki et al., J. Immunol. 169: 4136-46, 2002; Saneyoshi et al., Nature 417: 295-9, 2002; Neilson et al., Curr Opin Immunol. 13: 346-50, 2001; Diakos et al., Transplant Proc 33: 197-8, 2001; Behrens et al., Proc Natl Acad Sci USA 98: 1769-74, 2001; Abbott et al., Mol Biol Cell 9: 2905-16, 1998; Klein-Hessling et al., Proc Natl Acad Sci USA 93: 15311-6, 1996; and Timmerman et al., Nature 383: 837-40, 1996.

For example, similar to the first screening step, modulation of expression of an NF-AT responsive gene can be examined in a cell-based system by transient or stable transfection of an expression vector into cultured cell lines. Assay vectors bearing an NF-AT recognition sequence operably linked to reporter genes can be transfected into any mammalian cell line (e.g., HEK 293 cells as described in the Examples) for assays of promoter activity. General methods of cell culture, transfection, and reporter gene assay have been described in the art, e.g., Ausubel, supra; and Transfection Guide, Promega Corporation, Madison, Wis. (1998). Any readily transfectable mammalian cell line may be used to assay NF-AT promoter, e.g., HCT116, HEK 293, MCF-7, and HepG2 cells.

Constructs containing an NF-AT recognition sequence (or a transcription regulatory element of an NF-AT gene) operably linked to a reporter gene can be prepared using only routinely practiced techniques and methods of molecular biology (see, e.g., Sambrook et al. and Ausubel et al., supra). Alternatively, expression vectors containing a reporter gene under the control of NF-AT response elements can also be obtained commercially (e.g., from Clontech, Palo Alto, Calif.; see the Example below). NF-AT binding sites have been found in nearly all lymphokine promoters that are activated upon T cell activation. Examples of NF-AT responsive genes include IL-2 (Serfling et al., Biochim. Biophys. Acta 1263: 181-200, 1995; and Randak et al., EMBO J. 9: 2529-36, 1990), IL-4 (Rooney et al., Immunity 2: 473-83, 1995; and De Boer et al., Int'l J. Biochem. Cell Biol. 31: 1221-36, 1999), IL-5 (Prieschl et al., J. Immunol. 154: 6112-9, 1995), IFN-γ (Sica et al., J. Biol. Chem. 272: 30412-20, 1997), IL-3 (Masuda et al., Mol. Cell. Biol. 13: 7399-7407), granulocyte/macrophage colony-stimulation factor (GM-CSF; Duncliffe et al., Immunity 6: 175-85, 1997), and tumor necrosis factor α (Tsai et al., Mol. Cell. Biol. 16: 459-67, 1996; and Tsai et al., Mol. Cell. Biol. 16: 5232-44, 1996). Other than lymphokine and cytokine genes, NF-AT binding sites have also been found in other genes, e.g., CD25/IL-2 receptor α (Schuh et al., J. Exp. Med. 188: 1369-73, 1998), transcription factor Egr2 (Latinis et al., J. Biol. Chem. 272: 31427-34, 1997), transcription factor Egr3 (Mittlestadt et al., Mol. Cell. Biol. 18: 3744-51, 1998). Often, the NF-AT binding sites comprise a composite NF-AT+AP-1 recognition sequence. Composite NF-AT+AP-1 binding sites have been identified in a large number of diverse promoters and enhancers, e.g., promoters of cytokine genes and their receptors, promoters of genes for AP-1 family members, Ca²⁺-binding proteins, and other components of the regulatory network controlling cell cycle and apoptosis (see, e.g., Kel et al., J. Mol. Biol. 288: 353-376, 1999; and Serfling et al., Biochim Biophys Acta 1498: 1-18, 2000).

Any of these transcription regulatory sequences can be employed in the present invention to study a test agent's ability to modulate the transcription-regulating function of NF-ATs. When the test agent is assayed for ability to modulate expression level of an NF-AT gene, transcription regulatory elements of the NF-AT genes can be used in the screening assay. Transcription regulatory elements of the NF-AT genes have also been well known and characterized in the art, e.g., as disclosed in McCaffrey et al., J. Biol. Chem. 268: 3747-52, 1993; Hoey et al., Immunity 2: 461-72, 1995; and Northrop et al., Nature 369: 497-502, 1994.

When inserted into the appropriate host cell, the transcription regulatory elements in the expression vector induce transcription of the reporter gene by host RNA polymerases. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP).

Transcription driven by NF-AT response elements may also be detected by directly measuring the amount of RNA transcribed from the reporter gene. In these embodiments, the reporter gene may be any transcribable nucleic acid of known sequence that is not otherwise expressed by the host cell. RNA expressed from constructs containing an NF-AT response element may be analyzed by techniques known in the art, e.g., reverse transcription and amplification of mRNA, isolation of total RNA or poly A⁺ RNA, northern blotting, dot blotting, in situ hybridization, RNase protection, primer extension, high density polynucleotide array technology and the like. These techniques are all well known and routinely practiced in the art.

In addition to reporter genes, vectors for assaying expression under the control of an NF-AT recognition sequence can also comprise elements necessary for propagation or maintenance in the host cell, and elements such as polyadenylation sequences and transcriptional terminators to increase expression of reporter genes or prevent cryptic transcriptional initiation elsewhere in the vector. Exemplary assay vectors are the pGL3 series of vectors (Promega, Madison, Wis.; U.S. Pat. No. 5,670,356), which include a polylinker sequence 5′ of a luciferase gene. NF-AT response elements may be inserted into the polylinker sequence and tested for luciferase activity in the appropriate host cell. Assay vectors may also comprise additional enhancer or promoter sequences, depending on whether the transcription regulatory elements are sufficient to drive transcription of the reporter genes. For example, in addition to the NF-AT recognition sequence, the expression vectors can contain additional promoter sequence such as a minimal promoter (e.g., a promoter derived from Herpes simplex virus thymidine kinase) as discussed in Example 1.

If the NF-AT transcription regulatory sequence in the vector does not contain transcription initiation elements, an assay vector such as pGL3-Promoter may be used. This vector has transcription initiation elements from the SV40 promoter. In such vectors, transcription initiates from a heterologous site but the rate of transcription is increased by the presence of linked NF-AT response elements.

Other than monitoring transcription-regulating activities of NF-ATs, a test agent that modulates an NF-AT-modulatory polypeptide can be further screened for ability to modulate an NF-AT responsive gene. As noted above, a great number of genes are known to be regulated by the NF-AT transcription factors (e.g., IL-2 or IL-4). Thus, test agents that modulate an NF-AT-modulatory polypeptide can be subject to further screening for ability to modulate expression of any of these NF-AT responsive genes.

IV. Modulation of NF-AT Activity In Vivo

The present invention provides compositions and methods for modulating activities of the NF-AT transcription factors in a cell, and for modulating cellular processes mediated by NF-ATs. As a consequence of the connection between the NF-AT transcription factors and lymphokine production as well as T cell differentiation, modulation of cellular levels or other bioactivities of the NF-AT transcription factors can lead to modulation of the cellular processes mediated by NF-ATs. Modulation by the NF-AT modulators of the present invention (polypeptides or other molecules) can act through a number of mechanisms. For example, in some methods, modulation of cellular activities by NF-AT modulators of the present invention is achieved through modulating transcription-regulating activities of an NF-AT, e.g., its binding to an NF-AT response element. The modulation can also be due to a decrease or an increase in the cellular level of NF-ATs. For example, expression of NF-AT may be decreased or increased by binding of an NF-AT modulator to its promoter sequence.

To modulate NF-AT activity in vivo, a cell can be contacted with any a number of the NF-AT modulators identified in accordance with the present invention. In some methods, a modulator of NF-AT of the present invention is introduced directly to a subject (e.g., a human, a mammal, or other non-human animal). In some methods, a polynucleotide encoding a modulator of NF-AT of the present invention is introduced by retroviral or other means (as detailed below). For example, polynucleotides with sequences shown in Table 1 or substantially identical sequences or their fragments can be used to modulate NF-AT activity in vivo.

Activities of NF-AT modulators of the present invention can be examined or further verified in vivo by employing transgenic animals. For example, transgenic animals with integrated NF-AT response elements can be used to assay modulation of NF-AT activities in vivo. Transgenic animals (e.g., transgenic mice) harboring NF-AT recognition sequences can be generated according to methods well known in the art. For example, techniques routinely used to create and screen for transgenic animals have been described in, e.g., see Bijvoet (1998) Hum. Mol. Genet. 7:53-62; Moreadith (1997) J. Mol. Med. 75:208-216; Tojo (1995) Cytotechnology 19:161-165; Mudgett (1995) Methods Mol. Biol. 48:167-184; Longo (1997) Transgenic Res. 6:321-328; U.S. Pat. Nos. 5,616,491 (Mak, et al.); 5,464,764; 5,631,153; 5,487,992; 5,627,059; 5,272,071; and, WO 91/09955, WO 93/09222, WO 96/29411, WO 95/31560, and WO 91/12650.

In some embodiments, NF-AT recognition sequences operably linked to a reporter gene are injected into the embryo of a developing animal (typically a mouse) to generate a transgenic animal. Once integration of the transgene has been verified, tissues of the animal (e.g., lymphoid tissues) are then assayed for expression of the transgene. For example, where the NF-AT recognition sequence is linked to a reporter gene, tissues of the transgenic animal may be assayed either for reporter gene RNA or for the enzymatic activity of the reporter polypeptide.

In the transgenic animals, NF-AT recognition sequences will generally display appropriate regulation regardless of the site of transgene integration. However, constructs comprising the regulatory sequences can also be flanked by insulator elements to ensure complete independence from position effects (see Bell et al., Science 291:447-50, 2001).

V. Therapeutic Applications

The invention provides therapeutic compositions and methods for preventing or treating diseases and conditions due to abnormal cellular level or other biological activities of NF-AT. The NF-AT transcription factors play important roles in regulating lymphokine production and T cell differentiation. Many clinical conditions or disease states are linked to abnormal immune activities mediated by the T cells. For example, a subject having a neoplastic disease (e.g., lymphocytic leukemia) or T cell-mediated immune response may exhibit an amount of NF-AT protein or mRNA in a cell or tissue sample that is higher than the range of concentrations that characterize normal, undiseased subjects.

Thus, compositions containing a therapeutically effective dosage of an NF-AT modulator can be administered to a subject for treatment of immune diseases such as lymphocytic leukemias (e.g., T cell leukemia or lymphoma), transplant rejection reactions, and hyperactive or hypoactive T cell conditions. The pharmaceutical compositions can comprise a polypeptide modulator of NF-AT identified in accordance with the present invention (e.g., as shown in Table 1), an antibody against such modulators, or other modulators disclosed herein which directly or indirectly modulate NF-AT activities. In addition to treating these diseases or conditions, modulation of NF-AT activity or cellular levels is also useful for preventing or modulating the development of such diseases or disorders in a subject suspected of being, or known to be, prone to such diseases or disorders.

Other diseases and conditions are also known in the art which have implicated abnormal NF-AT activities. For example, NF-AT pathway is implicated in susceptibility to T cell-mediated injury in immune complex disease (Suzuki et al., J. Immunol. 169: 4136-46, 2002). Diseases that can be treated with the therapeutic compositions of the present invention further include autoimmune disease, e.g., arthritis wherein NF-AT activity contributes to disease processes.

A. Pharmaceutical Compositions

The NF-AT modulators of the present invention can be directly administered under sterile conditions to the subject to be treated. The modulators can be administered alone or as the active ingredient of a pharmaceutical composition. Therapeutic composition of the present invention can also be combined with or used in association with other therapeutic agents.

Pharmaceutical compositions of the present invention typically comprise at least one active ingredient together with one or more acceptable carriers thereof. Pharmaceutically carriers enhance or stabilize the composition, or to facilitate preparation of the composition. Pharmaceutically acceptable carriers are determined in part by the particular composition being administered (e.g., nucleic acid, protein, or modulatory compounds), as well as by the particular method used to administer the composition. They should also be both pharmaceutically and physiologically acceptable in the sense of being compatible with the other ingredients and not injurious to the subject. This carrier may take a wide variety of forms depending on the form of preparation desired for administration, e.g., oral, sublingual, rectal, nasal, or parenteral. For example, the NF-AT modulator can be complexed with carrier proteins such as ovalbumin or serum albumin prior to their administration in order to enhance stability or pharmacological properties.

There are a wide variety of suitable formulations of pharmaceutical compositions of the present invention (see, e.g., Remington: The Science and Practice of Pharmacy, Mack Publishing Co., 20^{th} ed., 2000). Without limitation, they include syrup, water, isotonic saline solution, 5% dextrose in water or buffered sodium or ammonium acetate solution, oils, glycerin, alcohols, flavoring agents, preservatives, coloring agents starches, sugars, diluents, granulating agents, lubricants, and binders, among others. The carrier may also include a sustained release material such as glyceryl monostearate or glyceryl distearate, alone or with a wax.

The pharmaceutical compositions can be prepared in various forms, such as granules, tablets, pills, suppositories, capsules, suspensions, salves, lotions and the like. The concentration of therapeutically active compound in the formulation may vary from about 0.1-100% by weight. Therapeutic formulations are prepared by any methods well known in the art of pharmacy. See, e.g., Gilman et al., eds., Goodman and Gilman's: The Pharmacological Bases of Therapeutics, 8th ed., Pergamon Press, 1990; Remington: The Science and Practice of Pharmacy, Mack Publishing Co., 20^{th} ed., 2000; Avis et al., eds., Pharmaceutical Dosage Forms: Parenteral Medications, published by Marcel Dekker, Inc., N.Y., 1993; and Lieberman et al., eds., Pharmaceutical Dosage Forms: Disperse Systems, published by Marcel Dekker, Inc., N.Y., 1990.

B. Dosages and Modes of Administration

The therapeutic formulations can be delivered by any effective means that could be used for treatment. Depending on the specific NF-AT modulators to be administered, the suitable means include oral, rectal, vaginal, nasal, pulmonary administration, or parenteral (including subcutaneous, intramuscular, intravenous and intradermal) infusion into the bloodstream.

For parenteral administration, NF-AT modulators (including polynucleotides encoding NF-AT modulators) of the present invention may be formulated in a variety of ways. Aqueous solutions of the modulators may be encapsulated in polymeric beads, liposomes, nanoparticles or other injectable depot formulations known to those of skill in the art. The nucleic acids may also be encapsulated in a viral coat.

Additionally, the compounds of the present invention may also be administered encapsulated in liposomes. The compositions, depending upon its solubility, may be present both in the aqueous layer and in the lipidic layer, or in what is generally termed a liposomic suspension. The hydrophobic layer, generally but not exclusively, comprises phospholipids such as lecithin and sphingomyelin, steroids such as cholesterol, more or less ionic surfactants such as a diacetylphosphate, stearylamine, or phosphatidic acid, and/or other materials of a hydrophobic nature.

The compositions may be supplemented by active pharmaceutical ingredients, where desired. Optional antibacterial, antiseptic, and antioxidant agents may also be present in the compositions where they will perform their ordinary functions.

The therapeutic formulations can conveniently be presented in unit dosage form and administered in a suitable therapeutic dose. A suitable therapeutic dose can be determined by any of the well-known methods such as clinical studies on mammalian species to determine maximum tolerable dose and on normal human subjects to determine safe dosage. Except under certain circumstances when higher dosages may be required, the preferred dosage of an NF-AT modulator usually lies within the range of from about 0.001 to about 1000 mg, more usually from about 0.01 to about 500 mg per day.

The preferred dosage and mode of administration of an NF-AT modulator can vary for different subjects, depending upon factors that can be individually reviewed by the treating physician, such as the condition or conditions to be treated, the choice of composition to be administered, including the particular NF-AT modulator, the age, weight, and response of the individual subject, the severity of the subject's symptoms, and the chosen route of administration. As a general rule, the quantity of an NF-AT modulator administered is the smallest dosage that effectively and reliably prevents or minimizes the conditions of the subjects. Therefore, the above dosage ranges are intended to provide general guidance and support for the teachings herein, but are not intended to limit the scope of the invention.

In some applications, a first NF-AT modulator is used in combination with a second NF-AT modulator in order to modulate NF-AT molecules to a more extensive degree than cannot be achieved when one NF-AT modulator is used individually.

C. Delivery of Polynucleotides Encoding NF-AT Modulators

In some methods of the present invention, polynucleotides encoding NF-AT modulators of the present invention (e.g., those listed in Table 1, substantially identical sequences, or fragments thereof) are transfected into cells for therapeutic purposes in vitro and in vivo. These polynucleotides can be inserted into any of a number of well-known vectors for the transfection of target cells and organisms as described below. The nucleic acids are transfected into cells, ex vivo or in vivo, through the interaction of the vector and the target cell. The compositions are administered to a subject in an amount sufficient to elicit a therapeutic response in the subject.

Such gene therapy procedures have been used to correct acquired and inherited genetic defects, cancer, and viral infection in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Felgner, TIBTECH 11:211-217 (1993); Mitani & Caskey, TIBTECH 11:162-166 (1993); Mulligan, Science 926-932 (1993); Dillon, TIBTECH 11: 167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Böhm eds., 1995); and Yu et al., Gene Therapy 1:13-26 (1994)).

Delivery of the gene or genetic material into the cell is the first step in gene therapy treatment of disease. A large number of delivery methods are well known to those of skill in the art. Preferably, the polynucleotides are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355, and lipofection reagents are sold commercially (e.g., Transfectam™ and Lipofectin™). Cationic and neutral lipids that are suitable for efficient receptor-recognition lipofection of polynucleotides include those described in Felgner, WO 91/17424, and WO 91/16024. Delivery can directed to cells (ex vivo administration) or target tissues (in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to subjects (in vivo) or they can be used to treat cells in vitro and the modified cells are administered to subjects (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In particular, a number of viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent.

pLASN and MFG-S are examples are retroviral vectors that have been used in clinical trials (Dunbar et al., Blood 85:3048-305 (1995); Kohn et al., Nat. Med. 1:1017-102 (1995); Malech et al., Proc. Natl. Acad. Sci. U.S.A. 94:22 12133-12138 (1997)). PA317/pLASN was the first therapeutic vector used in a gene therapy trial (Blaese et al., Science 270:475-480 (1995)). Transduction efficiencies of 50% or greater have been observed for MFG-S packaged vectors (Ellem et al., Immunol Immunother. 44(1):10-20 (1997); and Dranoff et al., Hum. Gene Ther. 1:111-2 (1997)).

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the outer surface. The ligand is chosen to have affinity for a receptor known to be present on the cell type of interest. For example, Han et al., Proc. Natl. Acad. Sci. U.S.A. 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual subject, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., a human subject). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from subjects).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

### EXAMPLES

The following example is provided to illustrate, but not to limit the present invention.

### Modulation of Expression from an NF-AT Enhancer Element

This Example describes identification of various NF-AT-modulatory polypeptides that regulate expression of a reporter gene under the control of an NF-AT recognition sequence. NF-AT recognition sequences control transcription of a great number of genes regulated by NF-AT (NF-AT responsive genes).

A. Generation of Arrayed cDNA Expression Library for Cell Based Screening

The Mammalian Genome Collection (MGC) was purchased from both Incyte Genomics (Palo Alto, Calif.) and ATCC (Manassas, Va.). This collection contained mouse and human cDNAs in a variety of vectors. These clones were arrayed into a 384 well plate format from the master collection using a QBot (Genetix, LTD, United Kingdom). The arrayed clones were stored as glycerol stocks which were used to inoculate 96-well deep well blocks (DWBs). The inoculated DWBs were grown overnight for 16 hr in Terrific Broth in a Hi-Gro (Gene Machines, San Francisco, Calif.). The cultures were centrifuged to pellet the bacteria.

The bacterial pellet was resuspended and DNA was prepared using a MWG RoboPrep 2500 (MWG Biotech AG, Ebersberg, Germany) and consumables from Macherey-Nagel (Duren, Germany). DNA was eluted directly into 96-well UV plates from Corning Costar (cat. #3635). DNA concentration was determined using a SPECTRAmax UV microplate reader (Molecular Devices, Sunnyvale, Calif.). DNA concentration was then normalized to 100 ng/μl on a MWG RoboSeq 4204 S. Normalized DNA in a 96-well format was compressed into 384-well plates, recreating the plate and well identities of the original arrayed 384-well glycerol stocks. Approximately 62.5 ng of DNA was spotted per well. The compression was done with a CCS Packard MiniTrak (Downers Grove, Ill.). Source plates for the compression were 96-well microtiter plates from Macherey-Nagel and destination plates were either white solid bottom Greiner 384 well plates (cat. #_{—}781073) or black clear bottom Greiner 384-well plates (cat. #_{—}781092). All destination plates utilized are tissue culture compatible.

B. Reporter Gene Vector and Transfection into Host Cells

pNFAT-TA-Luc is a member of the Mercury Pathway Profiling system, and was purchased from Clontech to monitor NFAT-mediated signal transduction pathways in mammalian cells by assaying for Luciferase activity. pNFAT-TA-Luc reporter vector contains three tandem copies of the NFAT consensus sequence (Fiering et al., Genes Devel. 4:1823-1834, 1990) and a minimal promoter derived from Herpes simplex virus thymidine kinase (HSV-TK) promoter.

Fugene 6 is a multi-component lipid-based transfection reagent purchased from Roche Molecular Biochemicals. Fugene 6 was combined with DNA at a ratio of 1:3 DNA(μg):Fugene 6(μl).

Human Embryonic Kidney cells (HEK 293 cells) obtained from American Tissue Culture Collection (Rockville Md.) were maintained in Dulbecco's Modified eagle Medium from Gibco, supplemented with 10% of Fetal Bovine Serum (Heat Inactivated). Penicillin (10,000u/ml), Streptomycin (10,000 ug/ml), and L-glutamine (29.2 mg/ml) from Gibco were added to the media. Cells were harvested with 0.05% Trypsin-0.53 mm EDTA (Gibco) at 80% confluency for the transfections.

Transfected cells were treated with 8 nm Phorbol 12-Myristate 13-Acetate (PMA, Sigma-Aldrich) for 6 hours prior to the Luciferase assay. The addition of PMA induces the binding of AP-1 transcription factor, which is required for NFAT reporter activation.

C. Transfection of MGC cDNAs into HEK 293 cells and Bright Glo Assay System

Mammalian Genome collection (MGC) 384 solid white plates were equilibrated to room temperature then briefly centrifuged for 3 minutes at 750 rpm in a Beckman Coulter Allegra centrifuge. Fugene 6 transfection reagent was added at 1:3 ratio (DNA:Fugene 6) of 0.3 μl/well to 20 μl/well of DMEM serum free media. pNFAT-TA-Luciferase reporter gene was added at 28.9 ng/well. The DNA/Fugene 6 complex was dispensed into the MGC 384 well plates immediately using the multidrop (Titertrek). Plates were incubated at room temperature for 45 minutes. HEK 293 cells were dispensed by multidrop at 2000 cells/well in 20 ul/well of DMEM in 3% Fetal Bovine Serum final concentration plus Penicillin/Streptomycin/Glutamine. Transfected cells were incubated at 37C, 5% CO₂ for 48 hours then assayed for Luciferase activity.

Transfected MGC plates were induced with PMA on the day of the assay for 6 hours. Then equal volume of Bright Glo (Promega, Madison, Wis.) was added to each well. The relative luminescence was quantitated using an Acquest 384.1536(LjL Biosystems Sunnyvale, Calif.) plate reader.

D. Results

Results from the above screens were normalized to a mean value. The most potent activators were identified as modulators of NF-AT. GenBank accession numbers of these modulators and the degree of up-regulation of the reporter gene expression by the modulators are shown in Table 1.

It is understood that the examples and embodiments described herein are for illustrative purposes only and that various modifications or changes in light thereof will be suggested to persons skilled in the art and are to be included within the spirit and purview of this application and scope of the appended claims. Although any methods and materials similar or equivalent to those described herein can be used in the practice or testing of the present invention, the preferred methods and materials are described.

All publications, GenBank sequences, patents and patent applications cited herein are hereby expressly incorporated by reference in their entirety and for all purposes as if each is individually so denoted.

1. A method for identifying an agent that modulates an NF-AT bioactivity, the method comprising:
(a) assaying a biological activity of an NF-AT-modulatory polypeptide encoded by a polynucleotide selected from the members listed in Table 1, or a fragment of said polypeptide, in the presence of a test agent to identify one or more modulating agents that modulate the biological activity of the polypeptide; and
(b) testing one or more of the modulating agents for ability to modulate an NF-AT bioactivity.

2. The method of claim 1, wherein (b) comprises testing the modulating agents for ability to modulate NF-AT in regulating expression of an NF-AT responsive gene.

3. The method of claim 1, wherein (b) comprises testing the modulating agents for ability to modulate cellular level of NF-AT.

4. The method of claim 1, wherein the NF-AT-modulatory polypeptide is a kinase and the biological activity is phosphorylation of a second polypeptide.

5. The method of claim 4, wherein the second polypeptide is NF-AT or a fragment of NF-AT.

6. The method of claim 1, wherein the NF-AT-modulatory polypeptide is a protease and the biological activity is proteolysis of a second polypeptide.

7. The method of claim 6, wherein the second polypeptide is NF-AT or a fragment of NF-AT.

8. The method of claim 1, wherein the test agent modulates cellular level of the NF-AT-modulatory polypeptide.

9. The method of claim 1, wherein the assaying of the biological activity of the NF-AT-modulatory polypeptide occurs in a cell.

10. The method of claim 9, wherein the NF-AT-modulatory polypeptide is expressed from said polynucleotide that has been introduced into the cell.

11. The method of claim 1, wherein the NF-AT bioactivity is inducing expression of a second polynucleotide that is operably linked to an NF-AT response element.

12. The method of claim 11, wherein the second polynucleotide encodes a reporter polypeptide.

13. The method of claim 12, wherein the testing for ability to modulate an NF-AT bioactivity comprises:
providing a cell or cell lysate that comprises the second polynucleotide that is operably linked to the NF-AT response element;
contacting the cell or cell lysate with the test agent; and
detecting an increase or decrease in expression of the second polynucleotide in the presence of the test agent compared to expression of the second polynucleotide in the absence of the test agent.

14. The method of claim 1, wherein the testing for ability to modulate an NF-AT bioactivity comprises contacting a cell or cell lysate with the test agent and determining cellular level of NF-AT or a fragment of NF-AT.

15. The method of claim 1, wherein the testing for ability to modulate the NF-AT bioactivity comprises contacting a cell or cell lysate with the test agent and determining ability of NF-AT to bind to a second polynucleotide that comprises an NF-AT response element in the cell or cell lysate.

16. A method for identifying an agent that modulates cellular level of NF-AT, the method comprising:
(a) assaying a biological activity of an NF-AT-modulatory polypeptide encoded by a polynucleotide selected from the members listed in Table 1, or a fragment of said polypeptide, in the presence of a test agent to identify a modulating agent that modulates the biological activity of the polypeptide; and
(b) testing the modulating agent for ability to modulate cellular level of NF-AT.

17. The method of claim 16, wherein the NF-AT-modulatory polypeptide is a transcription regulatory protein and the biological activity is transcription of a second polynucleotide.

18. The method of claim 17, wherein the second polynucleotide encodes an NF-AT or a fragment of the NF-AT.

19. The method of 16, wherein the testing comprises (i) contacting the modulating agent with a second polynucleotide operably linked to a transcription regulatory element of NF-AT; and (ii) detecting a change in cellular level of said second polynucleotide relative to cellular level of said second polynucleotide in the absence of the modulating agent.

20. The method of claim 19, wherein the contacting occurs in a cell.

21. The method of claim 19, wherein said second polynucleotide encodes a reporter polypeptide.

22. The method of claim 20, wherein said second polynucleotide encodes an NF-AT or a fragment of the NF-AT.

23. A method for identifying an agent that modulates expression of an NF-AT responsive gene, the method comprising:
(a) contacting a test agent with an NF-AT-modulatory polypeptide encoded by a polynucleotide selected from the members listed in Table 1;
(b) detecting a change in an activity of said NF-AT-modulatory polypeptide relative to the activity in the absence of the test agent; and
(c) detecting a change of expression level of the NF-AT responsive gene in the presence of the test agent identified in (b) relative to expression level of the NF-AT responsive gene in the absence of the test agent; thereby identifying the test agent as a modulator of expression of the NF-AT responsive gene.

24. The method of claim 23, wherein (a) and (b) are performed in a cell.
